(19) United States
(12) Patent Application Publication
(10) Pub. No.: US 2004/0171053 A1
(43) Pub. Date: Sep. 2, 2004

(54) Molecular microarrays and methods for production and use thereof

(76) Inventor: Celine Hu, Tiburon, CA (US)

Correspondence Address: MORRISON & FOERSTER LLP, 755 PAGE MILL RD, PALO ALTO, CA, 94304-1018, US

(21) Appl. No.: 10/794,305

(22) Filed: Mar. 5, 2004

Related U.S. Application Data
(63) Continuation of application No. 09/705,079, filed on Nov. 2, 2000, now abandoned.
(60) Provisional application No. 60/172,243, filed on Nov. 2, 1999.

(51) Int. Cl. C12Q 1/68; G01N 33/53; C12M 1/34

(52) U.S. Cl. 435/6; 435/7.1; 435/287.2

(57) ABSTRACT

The present invention provides microarrays comprising microparticles with known addresses, wherein the microparticles are coupled to chemical, biological, and/or cellular entities of interest. The invention also provided methods for producing microarrays.

### CROSS-REFERENCE TO RELATED APPLICATIONS

This application claims priority to U.S. Provisional Application Serial No. 60/172,243, filed Nov. 2, 1999, the disclosure of which is incorporated herein by reference in its entirety.

### STATEMENT OF RIGHTS TO INVENTIONS MADE UNDER FEDERALLY SPONSORED RESEARCH

Not applicable.

### TECHNICAL FIELD

This invention relates to microarrays which comprise ordered arrays of chemical, biological and/or cellular entities with known addresses. Specifically, this invention relates to microarrays comprising microparticles with known addresses, wherein the microparticles are coupled to chemical, biological and/or cellular entities of interest. These microarrays are useful, for example, in methods for analysis of gene expression, drug discovery and diagnostics. The invention also relates to methods for producing microarrays.

### BACKGROUND ART

There are several existing methods for the fabrication of surface-bound microarrays of molecules. In general, they fall into three approaches. The first is to synthesize the molecules directly on the microarray, with each molecule having a defined address. (Southern, R. L. et al. (1992) Genomics, 13:1008-1017.; Matson, R. S. et al. (1995) Analytical Biochemistry 224:110-116). For example, photolithographic technology and photosensitive protecting groups have been used for the synthesis of multiple biopolymers of distinct sequence in an array on a solid support. See (U.S. Pat. No. 5,445,934, Fodor et al. (1995); U.S. Pat. No. 5,510,270, Fodor (1996); U.S. Pat. No. 5,744,101, Fodor et al. (1998); U.S. Pat. No. 5,753,788, (1998) #262). One of the limitations of these methods is the accumulation of truncated polymers (i.e., “failure” sequences) generated from incomplete reactions in each coupling cycle. This limits both the attainable length and the purity of the polymers in the array.

A second approach is to deliver molecules to discrete sites on a solid substrate and immobilize them through covalent or noncovalent bonding. In a procedure referred to as “dot blotting,” molecules are delivered manually to specific sites on a solid support, for example, with micropipettes. Later methods were developed with improved delivery efficiency, such as those employing a 96 well microtiter plate format using either vacuum devices or pins to transfer materials. However, these practices are laborious and relatively coarse, being suitable only for forming limited quantities of arrays with relatively large spots.

More recently, robotic systems have been developed to deliver large numbers of samples in small volumes to form arrays. (U.S. Pat. No. 5,807,522, Brown 1998), describes a method for forming large quantities of microsize arrays by delivering molecules with a robotic dispensing device capable of depositing selected volumes between 0.002 and 2 nl of solution on a nonpermeable solid surface. This method requires highly sophisticated mechanical engineering, yet fails to solve many difficulties in achieving reproducible and highly uniform arrays. For example, it is difficult, using this approach, to precisely control the immobilization reaction at each spot, making it difficult to achieve highly reproducible arrays. One reason for this problem is that it is difficult to obtain or prepare a substrate that is chemically uniform across a relatively large surface, leading to inconsistency in chemical properties at different regions of the surface. In addition, because extremely small volumes are delivered at each spot, it is difficult to control evaporation, which affects reactant concentrations and, hence, reaction rate. It is also difficult to maintain a constant temperature across the entire surface, due in part to the chemical inconsistencies mentioned above. Thus, differential temperature effects on reaction rate can occur at different regions of the surface.

A third approach, described in (U.S. Pat. No. 5,605,662, Heller et al. 1997), involves the use of a programmable device with a plurality of electrodes, each of which can be charged either positively or negatively, thereby concentrating molecules of opposite charge and repelling molecules of like charge to enhance reaction efficiency and specificity. This device utilizes electrical fields to transport molecules to selected sites and to facilitate reactions, and was designed for automated performance of assays and reactions without manual intervention. In this approach, oligonucleotides labeled with aldehyde functional groups were concentrated at selected sites and covalently bound to a substrate by reaction with aminopropyltriethoxyl groups on the substrate. However, this device is designed to form one microarray at a time, and is more suitable for forming microarrays having a relatively small number of sites. Moreover, this approach fails to control the level of cross contamination, which contributes to background noise in test results. It also shares the problem of the approaches mentioned above in failing to provide means to achieve uniformity across the substrate, resulting in variability in test results.

U.S. Pat. No. 5,900,481 relates to compositions comprising at least one bead conjugated to a solid support and further conjugated to at least one nucleic acid.

The disclosures of all publications and patents cited herein are hereby incorporated by reference in their entirety.

### DISCLOSURE OF THE INVENTION

It is an object of the invention to provide methods and compositions for the construction of microarrays (e.g., of biological, chemical and/or cellular entities) which can be mass-produced and attain more uniform properties, higher purity, and molecular integrity than with present methods. It is an additional object of the invention to provide improved microarrays which can be more easily mass-produced, and which are characterized by greater uniformity, higher purity and molecular integrity.

Accordingly, the invention provides a microarray comprising: (a) a substrate, wherein the substrate is derivatized with either: i) a first compound comprising a first functional group, and at least one layer of a cross-linking compound comprising multiple second functional groups, or ii) a first compound comprising a first functional group, and a polymeric film comprising multiple second functional groups; and (b) either: i) a population of at least one entity of interest, wherein the population of at least one entity of interest is associated with a distinct address on the substrate through coupling of the entities and the second functional groups, or ii) a population of microparticles, wherein the population of microparticles has at least one entity of interest coupled thereto, and wherein the population of microparticles is associated with a distinct address on the substrate through coupling of the second functional groups with the microparticles, such that the at least one entity of interest occupies a distinct address on the substrate.

In another aspect of the invention, a microarray is provided, comprising: (a) a substrate; and (b) a population of microparticles, wherein the population of microparticles is associated with a distinct address on the substrate, and wherein the population of microparticles has at least one entity of interest coupled thereto, the at least one entity of interest being selected from the group consisting of polypeptides, carbohydrates, cells, hormones, ligands, amino acids, lipids, fatty acids, and small molecules; such that the at least one entity of interest occupies a distinct address on the substrate.

In another aspect of the invention, a microarray is provided, comprising: (a) a substrate; and (b) a population of microparticles, wherein each microparticle is less than 1 μm in diameter, wherein the population of microparticles is associated with a distinct address on the substrate, and wherein the population of microparticles has at least one entity of interest coupled thereto, such that the at least one entity of interest occupies a distinct address on the substrate.

In another aspect of the invention, the present invention provides a microarray produced by a method comprising: (a) providing a population of at least one entity of interest, wherein the entities are optionally coupled to microparticles; (b) providing a substrate, wherein the substrate is derivatized with an activatible compound capable of coupling to the entities of interest or to the optional microparticles; (c) contacting the population of entities with the substrate; and (d) activating the activatible compound at the desired location(s) on the substrate, such that the population of entities is coupled to the substrate in the desired location(s).

In another aspect of the invention, the present invention provides a method for constructing a microarray, wherein the method comprises: (a) providing a substrate, wherein the substrate is derivatized with either: i) a first compound comprising a first functional group, and at least one layer of a cross-linking compound comprising multiple second functional groups, or ii) a first compound comprising a first functional group, and a polymeric film comprising multiple second functional groups; (b) providing either: i) a population of at least one entity of interest, or ii) a population of microparticles, wherein the population of microparticles has at least one entity of interest coupled thereto, (c) localizing the population of entities or microparticles to a distinct address on a substrate; and (d) associating the population of localized entities or microparticles to their distinct address on the substrate through coupling of the second functional groups to the entities of interest or to the microparticles.

In another aspect of the invention, the present invention provides a method for constructing a microarray, wherein the method comprises: (a) providing a population of at least one entity of interest, wherein the entities are optionally coupled to microparticles; (b) providing a substrate, wherein the substrate is derivatized with an activatible compound capable of coupling to the entities of interest or to the optional microparticles; (c) contacting the population of entities with the substrate; and (d) activating the activatible compound at the desired location(s) on the substrate, such that the population of entities is coupled to the substrate in the desired location(s).

In another aspect of the invention, the present invention provides a method of producing microarrays comprising nucleic acid sequences, comprising: (a) providing a first microarray comprising: (i) a first substrate; (ii) a first population of at least one nucleic acid sequence, wherein the at least one nucleic acid sequence comprises a first nucleic acid hybridization sequence at the distal end of the nucleic acid sequence, wherein the first population of nucleic acid sequence(s) is optionally coupled to microparticle(s), and wherein the population of nucleic acid sequence(s) is associated with a distinct address on the first substrate; (b) providing a second microarray comprising: (i) a second substrate; (ii) a population of second hybridization sequence(s), wherein the second hybridization sequence(s) is complementary to the first hybrization sequence(s), wherein the second population of hybridization sequence(s) are optionally coupled to microparticle(s), and wherein the population of hybridization sequence(s) is associated with a distinct address on the second substrate; (c) contacting the first and second microarrays under hybridizing conditions, such that the first and second hybridization sequences hybridize; (d) exposing the hybridized first and second microarrays to nucleotide polymerizing conditions, such that said at least one nucleic acid sequence from the first microarray is used as a template for the production of a complementary nucleic acid sequence on the second microarray.

In another aspect of the invention, the present invention provides a method for producing multiple copies of a microarray on a single substrate, wherein the method comprises: (a) providing a population of microparticles, wherein the population of microparticles has at least one entity of interest coupled thereto; (b) providing a substrate for multiple copies of a microarray; (c) localizing the population of microparticles to the substrate at the desired location(s) for each microarray to be produced on the substrate; and (d) associating the population of microparticles to the substrate at the desired location(s) for each microarray to be produced on the substrate.

### BRIEF DESCRIPTION OF THE DRAWINGS

FIG. 1 is a graph showing an exemplary apparatus for the synthesis of a microarray using microparticles and microfluidics technology.

### MODES FOR CARRYING OUT THE INVENTION

### Definitions

For the purposes of the invention, the terms “substrate,” “support” and “surface” are used interchangeably herein to denote a material upon which an array is constructed.

For the purposes of the invention, an “address” is a unique location on a substrate which can be distinguished from other unique locations.

As used herein, “population of microparticles” refers to one or more microparticles.

As used herein, “population of at least one entity of interest” refers to one or more entities of interest.

As used herein, “entity of interest” refers to a population of molecules or cells of a single type, e.g., a polynucleotide or a polypeptide. Types of molecules which may be used in the invention include biological or chemical compounds, such as, for example, a simple or complex organic or inorganic molecule. A vast array of molecules can be synthesized, for example oligomers, such as oligopeptides and oligonucleotides, and synthetic organic compounds based on various core structures. In addition, various natural sources can provide molecules for the invention, such as plant or animal extracts, and the like.

The terms “polypeptide”, “oligopeptide”, “peptide” and “protein” are used interchangeably herein to refer to polymers of amino acids of any length. The polymer may be linear or branched, it may comprise modified amino acids, and it may be assembled into a complex of more than one polypeptide chain. The terms also encompass an amino acid polymer that has been modified naturally or by intervention; for example, by disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), peptide-like compounds, for example, peptoids, as well as other modifications known in the art.

The terms “polynucleotide”, “oligonucleotide”, and “nucleic acid” are used interchangeably herein to refer to polymers of nucleotides of any length. It also includes analogues and derivatives of oligonucleotides known in the art, such as, for example, 2′ O-methyl-ribonucleotides.

The terms “polysaccharide” and “carbohydrate” are used interchangeably herein.

“A”, “an” and “the” include plural references unless the context clearly dictates otherwise.

Chemical terms, unless otherwise defined, are used as known in the art.

### General Techniques

The practice of the invention will employ, unless otherwise indicated, conventional techniques in photolithography, microfluidics, organic chemistry, biochemistry, oligonucleotide synthesis and modification, bioconjugate chemistry, nucleic acid hybridization, molecular biology, microbiology, genetics, recombinant DNA, and related fields as are within the skill of the art. The techniques are described in the references cited herein and are fully explained in the literature. For molecular biology and recombinant DNA techniques, see, for example, (Maniatis, T. et al. (1982). Molecular Cloning: A Laboratory Manual, Cold Spring Harbor; Ausubel, F. M. (1987). Current Protocols in Molecular Biology, Greene Pub. Associates and Wiley-Interscience; Ausubel, F. M. (1989). Short Protocols in Molecular Biology: A Compendium of Methods from Current Protocols in Molecular Biology, Greene Pub. Associates and Wiley-Interscience; Sambrook, J. et al. (1989). Molecular Cloning: A Laboratory Manual, Cold Spring Harbor; Innis, M. A. (1990). PCR Protocols: A Guide to Methods and Applications, Academic Press; Ausubel, F. M. (1992). Short Protocols in Molecular Biology: A Compendium of Methods from Current Protocols in Molecular Biology, Greene Pub. Associates; Ausubel, F. M. (1995). Short Protocols in Molecular Biology: A Compendium of Methods from Current Protocols in Molecular Biology, Greene Pub. Associates; Innis, M. A. et al. (1995). PCR Strategies, Academic Press; Ausubel, F. M. (1999). Short Protocols in Molecular Biology: A Compendium of Methods from Current Protocols in Molecular Biology, Wiley, and annual updates; Sninsky, J. J. et al. (1999). PCR Applications: Protocols for Functional Genomics, Academic Press). For DNA synthesis techniques and nucleic acids chemistry, see for example, (Gait, M. J. (1985). Oligonucleotide Synthesis: A Practical Approach, IRL Press; Gait, M. J. (1990). Oligonucleotide Synthesis: A Practical Approach, IRL Press; Eckstein, F. (1991). Oligonucleotides and Analogues: A Practical Approach, IRL Press; Adams, R. L. et al. (1992). The Biochemistry of the Nucleic Acids, Chapman & Hall; Shabarova, Z. et al. (1994). Advanced Organic Chemistry of Nucleic Acids, Weinheim; Blackburn, G. M. et al. (1996). Nucleic Acids in Chemistry and Biology, Oxford University Press; Hermanson, G. T. (1996). Bioconjugate Techniques, Academic Press). For microfabrication, see for example, (Campbell, S. A. (1996). The Science and Engineering of Microelectronic Fabrication, Oxford University Press; Zaut, P. V. (1996). Micromicroarray Fabrication: a Practical Guide to Semiconductor Processing, Semiconductor Services; Madou, M. J. (1997). Fundamentals of Microfabrication, CRC Press; Rai-Choudhury, P. (1997). Handbook of Microlithography, Micromachining, & Microfabrication: Microlithography).

### Microarrays

The present invention provides microarrays in which distinct chemical, biological and/or cellular entities are associated with specific addresses on the surface of a substrate.

Accordingly, the invention provides a microarray comprising: (a) a substrate, wherein the substrate is derivatized with either: i) a first compound comprising a first functional group, and at least one layer of a cross-linking compound comprising multiple second functional groups, or ii) a first compound comprising a first functional group, and a polymeric film comprising multiple second functional groups; and (b) either: i) a population of at least one entity of interest, wherein the population of at least one entity of interest is associated with a distinct address on the substrate through coupling of the entities and the second functional groups, or ii) a population of microparticles, wherein the population of microparticles has at least one entity of interest coupled thereto, and wherein the population of microparticles is associated with a distinct address on the substrate through coupling of the second functional groups with the microparticles, such that the at least one entity of interest occupies a distinct address on the substrate.

In another aspect of the invention, a microarray is provided, comprising: (a) a substrate; and (b) a population of microparticles, wherein the population of microparticles is associated with a distinct address on the substrate, and wherein the population of microparticles has at least one entity of interest coupled thereto, the at least one entity of interest being selected from the group consisting of polypeptides, carbohydrates, cells, hormones, ligands, amino acids, lipids, fatty acids, and small molecules; such that the at least one entity of interest occupies a distinct address on the substrate.

In another aspect of the invention, a microarray is provided, comprising: (a) a substrate; and (b) a population of microparticles, wherein each microparticle is less than 1 μm in diameter, wherein the population of microparticles is associated with a distinct address on the substrate, and wherein the population of microparticles has at least one entity of interest coupled thereto, such that the at least one entity of interest occupies a distinct address on the substrate.

In another aspect of the invention, the present invention provides a microarray produced by a method comprising: (a) providing a population of at least one entity of interest, wherein the entities are optionally coupled to microparticles; (b) providing a substrate, wherein the substrate is derivatized with an activatible compound capable of coupling to the entities of interest or to the optional microparticles; (c) contacting the population of entities with the substrate; and (d) activating the activatible compound at the desired location(s) on the substrate, such that the population of entities is coupled to the substrate in the desired location(s).

Substrates

Substances that can be used for the formation of a substrate include any solid material which has the property or is capable of being derivatized to have the property of binding to microparticles, either covalently or noncovalently. Materials for use as a substrate include, but are not limited to, glass, silica, silicon, silicon dioxide, plastic, metal or ceramic, e.g., porcelain, as well as natural and synthetic polymers, such as, for example, cellulose, chitosan, dextran, polystyrene, and nylon. Any substance capable of forming a solid surface is appropriate for use as a substrate in the practice of the invention. The substrate can also consist of layers of different materials.

The substrate material is formed into a size and shape that is appropriate for the particular manufacturing process and application of the arrays. For example, in certain types of analysis in which high consumption of analytes and reagents is acceptable, it may be more economical to construct an array on a relatively large substrate (e.g., 1 cm×1 cm or larger), with the attendant advantage that a less sensitive and hence, more economical detection system can be employed. However, in many instances, limited quantities of analytes and/or reagents are available, creating a strong incentive to minimize consumption of these components. In these circumstances, smaller substrate sizes and smaller addresses are appropriate. In particular, since the size of an address can be as small as the size of a single microparticle (which can be on the order of 1-2 nanometers), the minimum substrate area will in some cases be determined by the number of addresses on the substrate.

In certain embodiments, a substrate will contain up to 10⁸ addresses, in other embodiments up to 10⁷, up to 10⁶, up to 10⁵, up to 10⁴, up to 10³ or up to 10² addresses.

An address can assume any shape that is compatible with the association of a microparticle with that address, and that allows the entity at each address to be distinguished (e.g., optically) from entities at all other addresses. The shape of an address can be, for example, circular, ovoid, square, rectangular or can comprise an irregular shape.

The size of each address will depend, among other things, on the size of the substrate, the number of addresses on a particular substrate, the quantities of analytes and/or reagents available, the size of the microparticles, and the degree of resolution required for any method in which the array is used. Sizes can range, for example, from 1-2 nanometers to several centimeters, but any size consistent with the application of the array is possible.

The spatial arrangement and shape of the addresses is designed to fit the particular application in which the microarray will be employed. Addresses can be closely-packed, widely dispersed or sub-grouped into a desired pattern suitable for a particular type of analysis.

In one embodiment, the surface of the substrate is derivatized with functional groups that couple to matching functional groups on a microparticle or on an entity designated for localization at a particular address on the array. A pair of reactive functional groups, in which one member of the pair forms a covalent bond with another member of the pair, can be used to associate a microparticle or an entity of interest to a substrate. In this case, one member of the pair is attached to the substrate, and the other to a microparticle or entity of interest for linkage to the substrate. Examples of suitable reactive functional group pairs include, but are not limited to, amino group/N-hydroxysuccinimide ester, sulfhydryl group/maleimide group, and carbonyl group/hydrazide. Additional examples can be found in various chemical catalogues, for example, that of the Pierce Chemical Co. In addition, pairs of reactive groups used in the production of chromatographic matrices, particularly those involved in affinity chromatography, as well as those used in protein and nucleic acid modification, are applicable to the invention. See, for example, (Means, G. E. et al. (1971). Chemical Modification of Proteins, Holden-Day; Sundaram, P. V. et al. (1978). Theory and Practice in Affinity Techniques, Academic Press; Wilchek, M. et al. (1984). Affinity Chromatography. Methods in Enzymology, Academic Press; Hermanson, G. T. et al. (1992). Immobilized Affinity Ligand Techniques, Academic Press).

In some embodiments, it is desirable to increase either the number or the accessibility of the functional groups on the surface of the substrate to facilitate the association and immobilization of the microparticles or entities of interest on the substrate (e.g., covalently immobilizing the microparticles or entities of interest onto a substrate with a higher density of functional groups results in an overall bond strength more able to withstand the mechanical forces applied in the processes required for various assay applications). This can be achieved by cross-linking compounds containing multiple functional groups onto the functional groups on the surface of the substrate. Any compound with suitable multiple functional groups may be used as a cross linking compound to amplify the total number of functional groups available, or to increase the the accessibility of the functional groups. Non limiting examples of suitable cross-linking compounds include, for example, polylysine, polyaspartate, polyglutamate, chitosan or copolymers such as, for example, polyserine-aspartate, etc. In one embodiment, the cross linking compound is polylysine. In one embodiment, the substrate is derivatized with one round of cross linking. In a preferred embodiment, the substrate is derivatized with two rounds of cross linking. In another preferred embodiments, the substrate is derivatized with at least three rounds, at least four rounds, at least 5 rounds of cross linking.

In one embodiment, the substrate surface can be either made of or coated by a polymeric film with very high concentration of functional, groups to achieve a suitable binding strength with the microparticles or entities of interest. These polymeric films can be formed by polymerizing pure monomers which contain functional groups (such as, for example, epoxy, amino, or carboxyl groups) or a mixture of different kinds of monomers resulting in high content of total number of functional groups. If the selected substrate surface has a low density of reactive functional groups (e.g., when the substrate is, for example, metal, glass, silica, ceramics, polystyrene or polypropylene), simply treating the substrate surface once with a multiple functional compound (such as is commonly done with polylysine), and attaching the microparticles, may not result in an overall bond strength sufficient to withstand the usual mechanical forces applied in the processes required for most assay applications. The application and reaction with a cross linking compound containing multiple functional groups in order to amplify the number of functional groups may be repeated as many times as needed to achieve the desired density of reactive functional groups. These amplification reagents containing multiple functional groups can be the same or different in each round.

This procedure amplifies the number of functional groups on the surface of the substrate when the number of functional groups introduced by the cross-linked compound outnumbers those consumed in the cross-linking reaction. This procedure can also increase the accessibility of the functional group if the cross-linked compound provides more space between the surface of the substrate and the functional group. For example, to increase the number of amino groups on the substrate, poly-lysine of suitable molecular weight can be cross-linked to the substrate. This step can be repeated to cross-link desired layers of poly-lysine, thus amplifying the number of amino groups and their distance from the surface.

Other chemical properties can be conferred to the substrate by incorporating other compounds with the desired property. For example, using poly-phenylalanine-lysine will confer more hydrophobicity than using poly-lysine. For hydrophilicity, polyethylene glycol of various sizes can be incorporated. There are various protecting groups and coupling chemistries that can be applied to achieve cross-linking in a controlled manner. Techniques and suitable protecting groups useful for this purpose are known in the field of peptide and oligonucleotide synthesis. See for example (Bodanszky, M. (1993). Peptide Chemistry: A Practical Textbook, Springer-Verlag; Bodanszky, M. (1993). Principles of Peptide Synthesis, Springer-Verlag; Bodanszky, M. et al. (1994). The Practice of Peptide Synthesis, Springer-Verlag; Fields, G. B. (1997). Solid-Phase Peptide Synthesis, Academic Press). For example, the amino groups on the poly-lysine can be protected with tert-butoxycarbonyl group (t-Boc). The terminal carboxyl group can react and be covalently linked with the original amino groups on the substrate by using a carbodiimide such as N,N′-dicyclohexylcarbodiimide or 1-ethyl-3-[3-Dimethylaminopropyl]carbodiimide with N-Hydroxysuccinimide as catalyst (see (Dierks, T. et al. (1992). Biochim Biophys Acta 1103(1):13-24; Sehgal, D. et al. (1994). Anal Biochem 218(1): 87-91)). If it is desirable, a limited number of amino groups on the poly-lysine can be converted to carboxyl groups by incorporating a small amount of an anhydride (such as, for example, succinic anhydride) in the protection reaction with N-(tert-butoxycarbonyloxy)succinimide, so that limited amounts of the amino groups on poly-lysine are converted to carboxyl groups. With more than one carboxyl groups on the t-Boc protected poly-lysine, it is easier to cross-link the polymer to the amino groups on the substrate. In a similar manner, to introduce more carboxyl groups, poly-aspartic acid or other polymers containing multiple carboxyl groups can be used. Protecting and coupling reagents can be selected to suit the requirements for the particular reaction. The cross-linking reaction can also be controlled through reactant concentrations, pH, temperature, etc. to effect the desired outcome.

Microparticles

Substances that can be used to form microparticles include any solid material that can be made into particles and that has the property or is capable of being derivatized to have the property of binding to a substrate and to the particular entities to be displayed on the microarray. The microparticles can be derivatized or non-derivatized. The binding of the microparticle to the substrate and to the entity of interest can be either covalent or noncovalent. Materials for use in the construction of microparticles include, but are not limited to, glass, silica, silicon, silicon dioxide, plastic, metal or ceramic, e.g., porcelain, as well as natural and synthetic polymers, such as, for example, cellulose, chitosan, dextran, polystyrene, and nylon. In one embodiment, the substrate is dervatized, and the microparticle is not derivatized. In another embodiment, the substrate is not derivatized, and the microparticle is derivatized. In yet another embodiment, both the substrate and the microparticle are derivatized.

Microparticles are available commercially from, for example, Bang Laboratories, Inc.; Seradyn, Inc.; Quantum Dot, Inc.; BioRad and Pharmacia, and can be obtained in various shapes and sizes. Any shape and/or size compatible with the desired use of the array is appropriate. Spherical microparticles are most commonly available. In one embodiment, spherical microparticles with a diameter between about 1 nm and 10 mm are suitable. In another embodiment, the microparticles are less than 1 μm in diameter. It is preferable that the microparticles are of a uniform size. However, if software programs known in the art are applied to normalize the signal strength verses the microparticle sizes, then size uniformity is not a critical requirement to yield consistent results in assay applications.

In one embodiment, microparticles with the desired entities on the surface are either ionic or magnetic in nature, thereby facilitating their initial localization to a specific address (see infra). Ionic properties can be furnished to a microparticle by, for example, derivatizing the microparticle with ionic groups, either positive or negative. Examples include, but are not limited to, carboxyl groups (providing negative charges) and amino groups (providing positive charges). Additionally, procedures used in the preparation of ion-exchange chromatography matrices can be applied to the preparation of charged microparticles. See, for example, (Kitchener, J. A. (1961). Ion Exchange Resins. 1st edition “reprinted with minor ammendments”, Methuen; Placek, C. (1970). Ion Exchange Resins, Noyes Data Corp.; Wilson, A. et al. (1981). Development and evaluation of ion-exchange resins for removal of specific metals in water treatment. Morgantown, Water Research Institute, Center for Extension and Continuing Ecucation, West Virginia University; Kunin, R. (1985). Ion Exchange Resins, R.E. Krieger Pub. Co.; Kunin, R. (1990). Ion Exchange Resins, R.E. Krieger Pub. Co.; International Conference on Ion Exchange (1991). New Developments in Ion Exchange: Materials, Fundamentals and Applications: Proceedings of the International Conference on Ion Exchange, ICIE '91; Philipp, W. H. et al. (1993). Ion Exchange Polymers and Method for Making Inventors, National Aeronautics and Space Administration.; Baumgartner, W. et al. (1997). Ion Exchange Resins, The Freedonia Group Inc.). In the synthesis of certain microparticles, an ionic co-polymer is incorporated in the initial polymerization step (e.g., those provided by Seradyn, Inc.), thereby imparting charge to the microparticle.

In another embodiment, ionic properties are imparted to a microparticle by virtue of the coupled molecule. For example, a microparticle comprising coupled nucleic acid will have a net negative charge at neutral pH. In addition, certain proteins and/or peptides, depending on their amino acid composition and the pH of the medium, exhibit a net positive or negative charge, as will be known to those of skill in the art.

Magnetic properties can be obtained by utilizing microparticles with paramagnetic materials embedded in the particle or attached to their surface. Any metal or substance capable of being magnetized is suitable for imparting magnetic properties to microparticles. Microparticles with magnetic properties are available commercially for example, from Dynal A. S. (Lake Success, N.Y. and Oslo, Norway) and Seradyn (Indianapolis, Ind.).

In one embodiment, a unique kind of chemical, biological or cellular entities are coupled to a microparticle. In one embodiment, a unique combination of chemical, biological and/or cellular entities are coupled to a microparticle. In another embodiment, the microarray has least two populations of microparticles or entities. In another embodiment, the microarray has at least 10, at least 100, at least 1000 populations of microparticles or entities. Methods described supra to functionalize the substrate for binding the microparticles are applicable for functionalizing the microparticle for coupling to the substrate or to the desired entities and are known to those skilled in the art. Additional examples are described infra.

Entities of Interest

Any biological, chemical, and/or cellular entity of interest that is capable of being coupled to a microparticle or to a substrate either covalently or non covalently can be used in the formation of a microarray according to the invention. These include, for example, biopolymers, small molecules, hormones, amino acids, lipids, ligands, fatty acids, nucleosides, nucleotides and nucleotide analogues (e.g., cAMP and cAMP derivatives) and include both synthetic and natural molecules. It also includes derivatives and analogues of the above. Cells or tissue samples can also be attached to a microparticle in the practice of the invention. These entities of interest need not be from a biological source; for example, products of combinatorial chemistry procedures can be coupled to microparticles in the practice of the invention. The chemical, biological and/or cellular entity can be attached to a microparticle or to a substrate through either a covalent or non-covalent linkage. In one embodiment, a population of at least one entity of interest is coupled directly to the substrate. In another embodiment, a population of microparticles are coupled to the substrate, wherein the population of microparticles are coupled to at least one entity of interest.

Biopolymers include polysaccharides, polypeptides and polynucleotides. Preferred biopolymers are polypeptides; more preferred are nucleic acid polymers. Nucleic acid polymers include, but are not limited to, oligonucleotides, polynucleotides, oligonucleotide and polynucleotide analogues, chimeric oligonucleotides and polynucleotides and modified nucleic acids. Nucleic acid polymers can be single-, double- or multiple-stranded.

In one embodiment, the at least one entity of interest is selected from the group consisting of polypeptides, carbohydrates, cells, hormones, ligands, amino acids, lipids, fatty acids, and small molecules. In a preferred embodiment, the at least one entity of interest is a nucleic acid. In separate embodiments, the at least one entity of interest is DNA or RNA. In another preferred embodiment, the at least one entity of interest is a polypeptide.

In one embodiment, two of more different types of chemical, biological and/or cellular entities are present on a single microarray. For example, many oncogenes are known to encode transcriptional regulatory proteins, which often interact both with regulatory nucleic acid sequences and additional regulatory proteins. Accordingly, an array comprising oligonucleotides, polypeptides and small molecules can be used, for example, to screen for molecules that interact with an oncogene product, to identify potential therapeutics. In another embodiment, an address may comprise at least one polypeptide and at least one nucleic acid.

Molecules can be chemically synthesized directly on the microparticles by methods known to those of skill in the art. For example, automated solid-phase peptide synthesis has been described by (Stewart, J. M. et al. (1984). Solid Phase Peptide Synthesis, Pierce Chemical Co.; Grant, G. A. (1992). Synthetic Peptides: A User's Guide, W. H. Freeman; Bodanszky, M. (1993). Principles of Peptide Synthesis, Springer-Verlag; Bodanszky, M. et al. (1994). The Practice of Peptide Synthesis, Springer-Verlag; Fields, G. B. (1997). Solid-Phase Peptide Synthesis, Academic Press; Pennington, M. W. et al. (1994). Peptide Synthesis Protocols, Humana Press; Fields, G. B. (1997). Solid-Phase Peptide Synthesis, Academic Press). Oligonucleotides can be prepared by automated chemical synthesis, using any of a number of commercially available DNA synthesizers, such as those provided by PE Biosystems. Compositions and methods for automated oligonucleotide synthesis are disclosed, for example, in (U.S. Pat. No. 4,415,732, Caruthers et al. (1983); U.S. Pat. No. 4,500,707 and Caruthers (1985); U.S. Pat. No. 4,668,777, Caruthers et al. (1987)).

In one embodiment, a collection of molecules synthesized by a combinatorial chemistry procedure (i.e., a library of compounds) can be coupled to microparticles or to the substrates, which are used to form a microarray for screening the molecules.

In another embodiment, tissue sections fresh, frozen or embedded in parafin, or cells harvested from cell culture can be coupled to microparticles or to the substrates. Cells grown within or on the surface of a microparticle, using fluidic bed methods as are known in the art, are also suitable. Using various cell origins and/or growth conditions, collections of microparticles representing various biological states can be constructed. For example, cells can be fixed to the microparticles, using standard fixation procedures, dehydration with alcohol or treatment with cross-linking reagents, to generate fixed cellular materials characteristic of a particular biological state. Exemplary fixation methods are described in (Bancroft, J. D. (1975). Histochemical Techniques, Butterworths; Troyer, H. (1980). Principles and Techniques of Histochemistry, Little Brown; Bancroft, J. D. et al. (1987). Enzyme Histochemistry, Oxford University Press; Sumner, B. E. H. (1988). Basic Histochemistry, Wiley; Lyon, H. (1991). Theory and Strategy in Histochemistry: a Guide to the Selection and Understanding of Techniques, Springer-Verlag; Graumann, W. et al. (1992). Histochemistry of Receptors, Gustav Fischer Verlag; Noorden, C. J. et al. (1992). Enzyme Histochemistry: A Laboratory Manual of Current Methods, Oxford University Press; Kiernan, J. A. (1999). Histological and Histochemical Methods: Theory and Practice, Butterworth Heinemann). Collections of microparticles, each collection containing a fixed cell population of a defined biological state, can be stored and used in the formation of microarrays, as described herein. One population of microparticles can be sufficient for making a large number of microarrays. In this way, the need to grow cells for each assay and the necessity of reproducing the exact growth conditions each time cells are grown is obviated. Instead, microparticles containing cellular materials from cells derived under identical conditions can be used in a great number of different assays. Furthermore, the use of microparticles containing cells can insure uniformity of the material being compared in different assays.

### Methods for Making Microarrays

Methods for the production of microarrays fabricated with arrays of selected entities are provided. In these and other methods, distinct chemical, biological and/or cellular entities to be displayed on the arrays may first be immobilized to microparticles in separate populations. The microparticles are then associated with various specific addresses on the substrate by one of several methods, to be described. Alternatively, the entities of interest are associated directly with the substrate.

In another aspect of the invention, the present invention provides a method for constructing a microarray, wherein the method comprises: (a) providing a substrate, wherein the substrate is derivatized with either: i) a first compound comprising a first functional group, and at least one layer of a cross-linking compound comprising multiple second functional groups, or ii) a first compound comprising a first functional group, and a polymeric film comprising multiple second functional groups; (b) providing either: i) a population of at least one entity of interest, or ii) a population of microparticles, wherein the population of microparticles has at least one entity of interest coupled thereto, (c) localizing the population of entities or microparticles to a distinct address on a substrate; and (d) associating the population of localized entities or microparticles to their distinct address on the substrate through coupling of the second functional groups to the entities of interest or to the microparticles.

In another aspect of the invention, the present invention provides a method for constructing a microarray, wherein the method comprises: (a) providing a population of at least one entity of interest, wherein the entities are optionally coupled to microparticles; (b) providing a substrate, wherein the substrate is derivatized with an activatible compound capable of coupling to the entities of interest or to the optional microparticles; (c) contacting the population of entities with the substrate; and (d) activating the activatible compound at the desired location(s) on the substrate, such that the population of entities is coupled to the substrate in the desired location(s).

In another aspect of the invention, the present invention provides a method of producing microarrays comprising nucleic acid sequences, comprising: (a) providing a first microarray comprising: (i) a first substrate; (ii) a first population of at least one nucleic acid sequence, wherein the at least one nucleic acid sequence comprises a first nucleic acid hybridization sequence at the distal end of the nucleic acid sequence, wherein the first population of nucleic acid sequence(s) is optionally coupled to microparticle(s), and wherein the population of nucleic acid sequence(s) is associated with a distinct address on the first substrate; (b) providing a second microarray comprising: (i) a second substrate; (ii) a population of second hybridization sequence(s), wherein the second hybridization sequence(s) is complementary to the first hybrization sequence(s), wherein the second population of hybridization sequence(s) are optionally coupled to microparticle(s), and wherein the population of hybridization sequence(s) is associated with a distinct address on the second substrate; (c) contacting the first and second microarrays under hybridizing conditions, such that the first and second hybridization sequences hybridize; (d) exposing the hybridized first and second microarrays to nucleotide polymerizing conditions, such that said at least one nucleic acid sequence from the first microarray is used as a template for the production of a complementary nucleic acid sequence on the second microarray.

In another aspect of the invention, the present invention provides a method for producing multiple copies of a microarray on a single substrate, wherein the method comprises: (a) providing a population of microparticles, wherein the population of microparticles has at least one entity of interest coupled thereto; (b) providing a substrate for multiple copies of a microarray; (c) localizing the population of microparticles to the substrate at the desired location(s) for each microarray to be produced on the substrate; and (d) associating the population of microparticles to the substrate at the desired location(s) for each microarray to be produced on the substrate.

Coupling of molecular or cellular entities to microparticles

Chemical, biological and/or cellular entities to be displayed on the arrays may first be coupled either covently or noncovalently to microparticles. Coupling may be conducted populationwise, such that each population of microparticles contains at least one entity of interest coupled thereto. There are numerous ways to couple molecules or cells to microparticles, using either chemical or biological means, see supra. Further examples are described infra.

Exemplary chemical methods for the coupling of chemical or biological entities to a microparticle include the coupling of a sulfhydryl group to a sulfhydryl, maleimide or iodoacetate group; carbodiimide-catalyzed coupling of an amino group to a succinimidyl ester, aldehyde or carboxyl group; coupling of a carbonyl group with a hydrazide group and non-specific coupling mediated by a photoreactive azide group. Additional coupling methods are known to those of skill in the art. Nucleic acids and peptides can be coupled by methods known in the art. See, for example, (Hermanson, G. T. et al. (1992). Immobilized Affinity Ligand Techniques, Academic Press; Shabarova, Z. et al. (1994). Advanced Organic Chemistry of Nucleic Acids, Weinheim; Hermanson, G. T. (1996). Bioconjugate Techniques, Academic Press). In one embodiment, the surface of a microparticle is treated with silane, to coat the surface of the particle with reactive groups (Joos, B. et al. (1997). Analytical Biochemistry 247: 96-101). Hydrophobic interactions and physical entrapment (e.g., with membrane, polymer or within a pore) can also be used.

Biological methods of attaching a molecule to a microparticle are based on specific biological interactions which include, but are not limited to, avidin-biotin; protein-ligand; antibody-antigen; antibody-hapten, sugar-lectin and specific interactions between complementary nucleic acids. Covalent or non covalent linkage of a molecule to a microparticle is also attainable through ligation and/or nucleic acid polymerization technologies. For example, oligonucleotides can be ligated to microparticles. Linker oligonucleotides or polynucleotides with sequences complementary to both the sequences of the molecules to be linked to the microparticles and those on the microparticles can be applied to bring the two sequences at a juxtaposition to be subsequently joined by a ligase. If it is preferable, linker molecules can be used that hybridize to both sequences on the microparticles and the molecules to be linked but instead of bringing them to a juxtaposition, leave a gap between the two sequences. Polymerase can be used to fill in the gap, and ligase then used to connect the two strands. Exemplary ligase enzymes include E. coli DNA ligase, T4 DNA ligase, Taq DNA ligase and T4 RNA ligase. Exemplary nucleic acid polymerases include E. coli DNA polymerase I (Pol I), the Klenow fragment of Pol I, Taq polymerase, T4 DNA polymerase, T7 DNA polymerase, E. coli RNA polymerase, T7 RNA polymerase, T3 RNA polymerase, and SP6 RNA polymerase. Additional procaryotic and eucaryotic ligases, DNA polymerases and RNA polymerases are known to those of skill in the art. Additionally, various reverse transcriptase enzymes, as known to those of skill in the art, can be used in the practice of the invention.

For enzymatic coupling of a single-stranded oligonucleotide or polynucleotide to a microparticle by polymerization, the template must be removed following polymerization. This can be accomplished by denaturation, using, for example, heat, high pH, organic solvents and/or enzymatic denaturation. If it is desired to couple a double-stranded oligonucleotide or polynucleotide to a microparticle, the template need not be removed. It can optionally be covalently coupled to the microparticle by methods known in the art and described supra. For example, functional groups such as aliphatic primary amino, or carboxyl groups can be linked to microparticles or a 3′ terminal ribose group can be introduced which upon oxidation by periodate can be linked to amine groups on the microparticles (see (Hermanson, G. T. et al. (1992). Immobilized Affinity Ligand Techniques, Academic Press)).

Association between microparticles and the substrate

Microparticles containing coupled entites of interest are associated with a substrate such that an ordered array of entities is formed. In one embodiment, this is achieved by synthesizing separate populations of microparticles, with each population having a distinct entity or a chosen mixture of entities coupled to the microparticles in that population. Each population is then associated with a unique address on the substrate, thereby placing a distinct entity or a chosen group of entities at each address on the substrate. Alternatively, a single microparticle, comprising a distinct entity coupled thereto, is associated with each address on the substrate. A mixture of microparticles synthesized in different batches coupled with different entities can be associated with one address.

The methods described herein for localizing and associating a microparticle to a substrate are also applicable for coupling an entity of interest to a substrate.

Microparticles containing coupled molecules or cells can be delivered to the surface of a substrate in the form of a liquid suspension of microparticles or as a dry powder.

One method for localization of a microparticle to an address utilizes ionic interactions between an address and a microparticle. For example, charged microparticles can be localized on a substrate in which a programmable electrode or electrical field is placed at each address. If the electrode at a particular address is positively charged, and the electrodes at all other addresses on the array are negatively charged, a negatively-charged microparticle (for example, a microparticle containing a coupled oligonucleotide) will be attracted to the address containing the positively-charged electrode and repelled from all other addresses. See U.S. Pat. No. 5,605,662 for additional details on the use of ionic properties for the localization of molecules on an array. Once a population of microparticles is localized to an address, excess microparticles can be recovered and the localized microparticles can be associated with the address (e.g., by covalent linkage); alternatively, the localization process can be repeated with a different population of microparticles at a different address. Because the microparticles physically occupy space at an address; once localized, they prevent additional microparticles from being in contact with the substrate at that address. Thus, it is possible to perform a number of cycles of localization per cycle of association to make the process more efficient. Sequential repetition of the localization and association process allows the construction of a microarray with a distinct species of microparticle (distinct by virtue of its coupled entities) localized at each unique address.

Localization of a microparticle to a specific address can also be achieved by utilizing magnetic properties of the array (e.g., a magnetic field) and the microparticles. For example, a microparticle can contain an iron core or be otherwise derivatized so as to possess magnetic properties. See supra. Each address on the substrate can be independently magnetized with designed integrated circuits. For example, each address can contain a metal core encircled by electrical wire; the direction of current in the wire will determine the magnetic polarity of the address. A microparticle having a particular magnetic charge can then be localized to an address having an opposite magnetic charge.

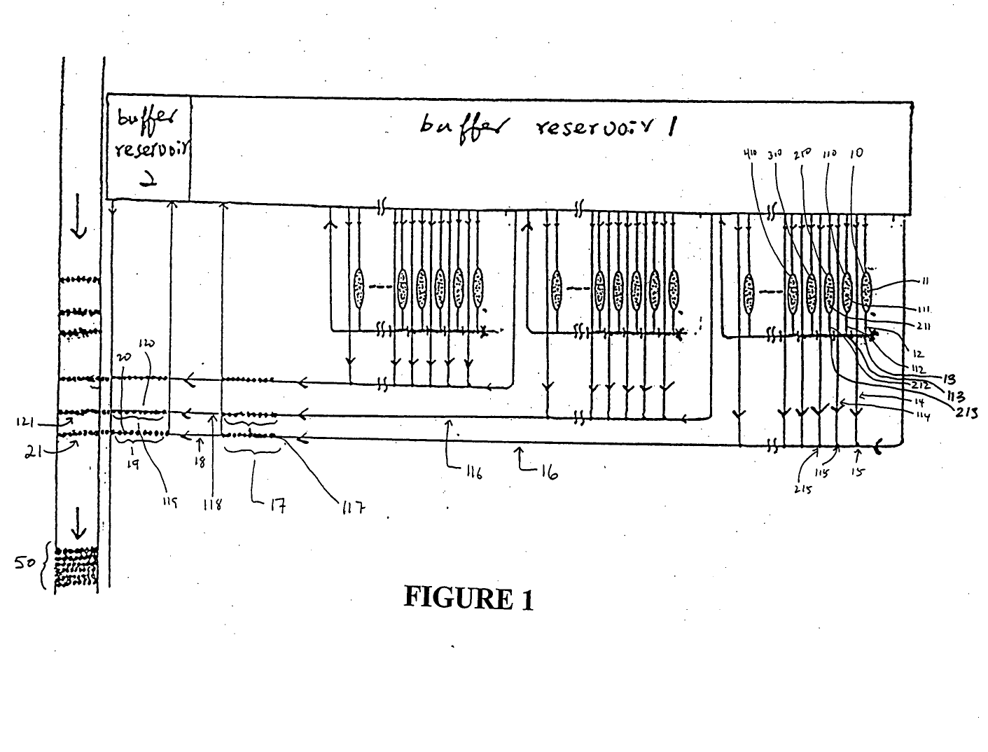

Localization can be achieved using various types of microfluidic technology, as are known in the art. See, for example, Service (1998) Science 282:399-401; U.S. Pat. No. 5,885,470 and references cited therein; and U.S. Pat. No. 5,932,315 and references cited therein. A non-limiting example for the assembly of a microarray using microfluidics is presented in Example 2, infra. Generally, an apparatus for use in the invention may comprise a plurality of microparticle reservoirs 10, 110, etc (FIG. 1). Each reservoir contains a population of microparticles 11, 111, etc., each bearing a distinct entity or group of entities. Microparticles are released from a reservoir into channel 12, 112, etc. and moved along the channel to site 13, 113, etc. Between each unique population of microparticles (i.e., between reservoirs 10 and 110, between channels 12 and 112, between sites 13 and 113), is a barrier which allows buffer to pass but retains the microparticles within their respective reservoirs, channels and sites.

Microparticles are moved individually or as a group, by microfluidics, from site 13 along a second channel 14 to site 15, and, from there, along channel 16 to site 17. At site 17, a group of populations of microparticles, each population containing a unique entity or group of entities, are aligned in close contact. The aligned microparticles are then moved along channel 18 to site 19, where the aligned microparticles are covalently or noncovalently cross-linked to one another to form a microparticle chain 20. Other microparticles chains (120, 220, etc.) can similarly be constructed. Pairs of reactive groups suitable for crosslinking of microparticles have been described supra, in connection with the discussion of linkage of microparticles to substrates and coupling of molecules to microparticles. For example, microparticles can be derivatized with biotin on their surface. At site 19, in the buffer reservoir 2, a suitable buffer containing avidin or a derivatized linker molecule with two or more avidins can be used to cross-link the microparticles. Similarly, chemical cross-linking can be initiated by using an appropriate buffer that effects change in pH, oxidation state or provides a catalyzing agent.

The microparticle chains 20, 120, etc. are then transported from sites 19, 119, etc. to sites 21, 121, etc. It is important to keep the chains extended and oriented in a fashion such that they can be stacked neatly. This can be accomplished electromagnetically by designing microsized programmable electrodes or magnetic cores in this area to guide the movement of these strings of microparticles to line up and stack in an orderly fashion.

Finally, the microparticle chains 20, 120, etc. are moved from sites 21, 121, etc. and deposited on the surface of a substrate 50, generating an array of addresses, wherein each address comprises a distinct entity or group of entities.

The process is repeated as often as desired, each time resulting in the deposition of a new array of microparticles on the substrate.

In a separate embodiment, “empty” microparticles, not bearing entities to be displayed, can be interspersed between microparticles which contain coupled molecules, thereby acting as spacers. This has the advantage of providing a physical barrier to minimize cross contamination between addresses.

Other methods for localizing the microparticles include, for example, dot blotting and robotic dispersion, as described supra, for example, in U.S. Pat. No. 5,807,522.

Following localization to a particular address, a microparticle is associated through either a covalent or a noncovalent linkage to that address. Noncovalent linkages can be established with binding interactions such as biotin and avidin, ligands and receptors, antigen and antibodies or hybridization interactions between nucleic acids with complementary sequences. A covalent linkage can be formed with either chemical or enzymatic methods. Formation of a covalent bond between an address and a microparticle can be accomplished through the use of particular pairs of reactive groups, as described supra, with one of the pair present at the address and the other on the microparticle. Reactive groups can be attached to the surface of a substrate and the microparticles as described supra. Covalent or noncovalent linkages can be formed between the substrate and the microparticles by applying molecular biology techniques using polymerases or ligases as described supra. With complementary sequences on the free ends of the nucleic acid molecules attached to both microparticle and the substrate, the linkage can be formed by hybridization. The hybridized sequence can be extended using either ligation or polymerization to enhance the bonding strength. The non-covalent bond of hybridized nucleic acids can be turned to covalent bonds by application of reagents such as psoralen (Kornhauser, A. et al (1982). Science 217: 733) that cross-link the two strands.

Association of microparticles to a substrate can be accomplished after any number of rounds of localization. Thus, localization of a single population of microparticle to a unique address can be followed by association, or a number of different populations of microparticles can be localized, each at a unique address, followed by association of all populations simultaneously.

In another embodiment of the present invention, a mask comprising orifice(s) corresponding to the desired location(s) for a particular population of microparticles is applied and an activatible group is used to link the molecular or cellular entities of interest (optionally coupled to microparticles) to the substrate. In one embodiment, the activatible group may comprise a photoreactive group. In another embodiment, the activatible group may comprise a heat activatible adhesive. In separate embodiments, optic fibers or micromirrors can also be substituted for the mask(s) in the methods of this invention for use in isolating specific regions of the substrate for activation.

For example, separate populations of microparticles may be coupled with unique chemical, biological and/or cellular entities of interest as described supra. Glass or silica plates or wafers can be derivatized with a high density of photoreactive groups, as described supra. Suitable photoreactive groups may be found, for example, in Pierce Catalog. Alternatively, plates or wafers may be coated with a polymeric film containing a high density of suitable functional groups such as amino, carboxyl or epoxy that can be used with or without further amplification and derivatization. When making the choice of applying polymeric films, one has to consider avoiding those that absorb energy at the same wavelength as the photoreactive groups. Modification to impart other characteristics such as hydrophilicity, hydrophobicity, positive or negative charges are known to those skilled in the art and as described in the references cited, see supra.

Microparticles (or entities of interest) are plated on the suitably derivatized surface. On one side of the plate or wafer, preferably the side of the wafer where the microparticles are not placed, a mask is applied, designed with orifice(s) corresponding to the desired location(s) for a particular population of microparticles. The photoreactive groups are activated by passing electromagnetic radiation of appropriate wavelength through the mask, thus initiating the coupling reaction to bind the microparticles to the plate. Further rounds of photoactivation using different masks and different populations of microparticles may be used to form an array of distinct populations of entities coupled to distinct addresses.

Heat activatible adhesives may also be used instead of photoactivatible groups. Nonlimiting examples of suitable heat activated adhesives, and methods for their use, are described in, for example, (Bonner, R. F. et al. (1997) Science, 278 (5342): p.1481-1483., Emmert-Buck, M. et al. (1996) Science, 274 (5289): 998-1001.) For example, separate populations of microparticles may be coupled with unique chemical, biological and/or cellular entities of interest as described supra. Glass or silica plates or wafers can be derivatized with a heat activatible adhesive capable of coupling to the microparticles. Microparticles are then plated on the suitably derivatized surface. On one side of the plate or wafer, a mask is applied, designed with orifice(s) corresponding to the desired location(s) for a particular population of microparticles. The heat activatible groups are activated by shining light of an appropriate wavelength through the mask, thus initiating the coupling reaction to bind the microparticles to the plate. Further rounds of adhesion using different masks and different populations of microparticles may be used to form an array of distinct populations of entities coupled to distinct addresses.

In these examples, the association of the population of microparticles to the substrate at a specific address is determined by the position of the orifice in the mask. There is little requirement for localization of the population of microparticles to a particular address before activation of the activatible groups. However, if desirable, microparticles with paramagnetic properties or net electric charges can be used and before the photoreactive coupling of the microparticles to the plate or wafer, a magnetic or electrical field can be applied to enhance or control the density of microparticles associated with the plate or wafer. In this case the magnetic or electric field can be applied evenly across the whole plate surface without having to provide features for individual addresses. After the association step, the microparticles that did not bind can be retrieved. The plate or wafer can be cleaned and ready for a second round of reaction with a different population of microparticles and a mask that directs them to associate at a second set of positions.

Multiple microarrays comprising microparticles may also be formed on a single substrate. Any of the methods disclosed herein may be used in the process of this invention. As a nonlimiting example, a mask comprising holes corresponding to the desired location(s) for each array to be produced on a single substrate may be constructed and contacted with the substrate. A population of microparticles is contacted with the substrate, and the desired locations on the substrate activated with light of an appropriate wavelength, thus coupling the microparticles to the desired location(s) for each array simultaneously. In this manner, large number of arrays can be formed on the plate or wafer and optionally subsequently cut into individual microarrays.

Microarrays comprising an array of nucleic acid sequences can be used as a template for the formation of additional microarrays, wherein the new microarrays comprise complements of the template microarray, as follows. A template microarray bearing a plurality of different single stranded nucleic acid sequences (optionally coupled to microparticles) are associated at distinct addresses. At the distal end of each sequence (i.e., the end farthest from the substrate), a short common sequence is present. A second array is constructed to contain a sequence complementary to the common sequence at each distinct address. The common sequences preferably are at least 5 nucleotides in length. In another embodiment, the common sequences are at least 10 nucleotides in length.

The two arrays are placed in contact with each other, under ionic and buffer conditions which favor hybridization between the common sequences and their complements. The common sequences may be identical or different for each address, as long as the appropriate complementary sequence is present on the second array. If necessary, various spacers, such as nucleotide homopolymers and/or polyethylene glycol linkers, can be interposed between the hybridizing sequences and the substrate, to facilitate interaction of the complementary sequences. It is preferred to have the individual addresses be relatively far apart in order to minimize cross-contamination.

After hybridization between the common sequences and their complements, the arrays, still in proximity, are subjected to conditions favoring nucleotide polymerization such as, for example, provision of a DNA polymerase and deoxynucleoside triphosphate substrates under appropriate conditions of pH, ionic strength and cation concentration, as are known to those of skill in the art.

Polymerization will generate an ordered microarray of complementary copies of the sequences present on the template microarray. The nucleic acid sequences on the two microarrays can be melted to produce the template microarray and a new complementary microarray. The two microarrays can be The process can be repeated to generate multiple copies of a specific microarray. In some cases, it may be more economical to produce microarrays in this fashion.

Placement of a plurality of discrete, distinguishable addresses on a substrate can be accomplished by any of a number of methods that are known to those of skill in the art. These include, for example, micromachining, microlithography, electron beam lithography, ion beam lithography, and molecular beam epitaxy, as known to those of skill in the art.

In some cases, it may be desirable to include an orientation marker on the microarray. This can be achieved by placing one or several microparticles, containing a readily identifiable signal (such as a chromophore or fluorophore) at one or more specific locations on the substrate. To make the orientation indicator(s) distinguishable from other addresses on the substrate, it (they) can be designed, for example, to be a distinct shape or color or a distinct combination of colors and/or shapes.

### Advantages

There are several significant advantages in first coupling molecules of interest to microparticles and then using these microparticles to form arrays.

First, the method of the invention includes an initial step of linking the molecules to the microparticles. This offers the advantage of achieving much more reproducible arrays. Since the immobilization of molecules to the microparticles is performed in one reaction vessel, the resulting linkage of molecules to each microparticle is very similar to that of any other microparticle in the same vessel. They can be subjected to quality control tests prior to applying them to the array. By contrast, it is difficult to achieve uniformity when association (immobilization) of the molecules must be performed separately at each individual address. In contrast, the linkage of microparticles to the substrate is formed through multiple bonds. All it requires is that the combined strength of these multiple bonds is above a threshold that is sufficient to hold the microparticles in place through the conditions required in the desired applications. The bonding strength can be in great excess and would not affect the uniformity of the arrays since the uniformity is determined by the uniformity of the coupling of the entities to the microparticles. The substrate surface variation among addresses is no longer as critical an issue with the present invention than it is when the entities of interest are linked directly to the substrate. For example, using direct linkage, the amount of entity coupled to the substrate is proportional to the reactivity at each address. Any variability contributes to the inconsistency among addresses and microarrays.

Second, certain prior approaches are limited with respect to the nature of linkages to the substrate. For example, the distribution and localization of molecules using electric fields with concurrent covalent linkage of the molecules directly to a substrate, as disclosed, for example, in U.S. Pat. No. 5,605,662, seriously limits the choice of chemical reactions that can be performed for associating a molecule with a substrate. The cross-linking reaction has to be performed under an electric field. It must not perturb nor be perturbed by the electric field nor can it add to the problem of electrolysis or be sensitive to it. There is also has a practical limit to the time for the reaction. Prolonged application of the electric field leads to accumulated problems with electrolysis which can damage the entities of interest that are to be included in the array. Because the molecular or cellular entities designated for inclusion in arrays may have greatly diverse chemical properties, preserving their chemical integrity throughout the process of association (immobilization) is of great importance for array function. For example, oligonucleotide bases must be capable of hybridizing to the analyte to serve as efficient probes, and hybridization ability is dependent on retention of functional groups on the bases. Proteins are even more sensitive to chemical manipulation than nucleic acids. The present invention, by separating the association procedure into two steps (first, linking the molecules of interest to the microparticles and then, forming the array with the microparticles) allows a much wider choice of conditions, both for association of the microparticle to the substrate and for the coupling of the molecule to the microparticle. The wider availability of reaction conditions for cross-linking makes it possible to design more optimal association conditions and achieve the desired end results such as proper density, preservation of the chemical integrity of the molecule, desired linking groups etc. This approach also overcomes the constraints associated with the use of a robotic dispenser to deliver small volumes of substance to each address, since this must be completed in a short time before the evaporation alters the reaction conditions drastically.

Third, the process of delivering entities to a substrate via microparticles offers the option that the association (immobilization) of microparticles to the substrate can utilize functional groups derivatized directly on the microparticles. The functional groups used for association do not have to be part of the entities which are to be displayed in the arrays. This means that a biopolymer, for example, does not need to possess a functional group to be used for association with the substrate. Instead, such an entity can be immobilized on a microparticle, for example, by non-covalent interactions such as hydrophobic interactions, or an entity can be physically entrapped in a microparticle. This, in turn, offers a better chance to preserve the chemical integrity of the entities to be displayed. It also offers greater flexibility in the types of chemistry available for association of the microparticles to the substrate. For example, the functional groups applied to the microparticle matrix and the substrates can be more chemically reactive than functional groups which can feasibly be applied to the entities of interest. It also offers the opportunity to design and form the microparticles and the substrate with chemical properties more suitable for the application of choice.

Fourth, the electrical charges on the microparticles used to localize them to their respective addresses with applied electric fields do not have to be imparted to the entities which are to be displayed on the arrays (i.e., it is not necessary, for a, e.g., biopolymer to be charged). The microparticles can be directly derivatized with the appropriate charge. This is of particular importance when the entities constituting the arrays do not bear a net charge.

Fifth, the invention minimizes the problem of contamination of a particular address on the microarray, during assembly of the array, by entities destined for other addresses. This is a difficult problem to control when an array is synthesized by introducing molecules in solution to the surface of a substrate, because there is a finite probability of a molecule binding non-specifically to a non-designated address. Even very low levels of nonspecific binding can lead to a high degree of cross-contamination after repeated cycles of binding. For example, a rate of non-specific binding of 0.5% per cycle will lead to significant contamination of an address after 200 cycles of localization. The presence of high amounts of the contaminating substances at each site can cause nonspecific signal resulting in high background noise. The invention minimizes problems of cross-contamination due to non-specific binding because, after localization, a species of microparticles occupies a defined physical space. Once a space, such as an address, is occupied, other particles are excluded from occupying that same space, thereby limiting the ability of a non-designated microparticle to contaminate a particular address. Moreover, it is easier to wash off the nonspecifically associated microparticles from the surface of the substrate compared to molecules. The shear mass of microparticles, compared to smaller molecules, even macromolecules such as nucleic acids or proteins, is much greater. It requires much more bonding strength between the substrate and a microparticle for it to stick than that of a small molecule or macromolecule. Therefore, the problem of nonspecific binding is minimized.

Sixth, unlike the microarrays produced by chemical synthesis of the displayed entities directly on the microarray, the molecules designated for display on the array can be purified to ensure that they are of suitable purity before being coupled to the microparticles.

### Applications

The micro arrays of the present invention may be used, for example, in diagnostics, forensics, drug discovery and development, molecular biology analysis (such as array-based nucleotide sequence analysis and array-based analyses of gene expression), protein property and function analysis, pharmacogenomics, proteomics and additional biological and chemical analyses.

### EXAMPLES

The following examples are provided to illustrate, but not to limit, the invention.

### Example 1

### Construction of a Microarray Using Masking and Photoreactive Methods

Separate populations of DNA containing a primary aliphatic amine group are prepared by automated solid phase synthesis or by performing PCR with one of the primer pair containing a primary aliphatic amine group. The DNA is then purified with Centricon (Amicon) filters of the appropriate molecular weight cut off. The purified cDNA with amino groups is linked to carboxylated microparticles (such as the ones by Seradyn or Bang Laboratories) using carbodiimide and N-hydroxylsuccinimide. Glass or silica wafers are derivatized with a high density of photoreactive groups, as follows. The wafer is treated with 3-aminopropyltrimethoxysilane solution to impart amino groups on the substrate (Joos, B. et al. (1997). Analytical Biochemistry 247:96-101). The number of amino groups is amplified by cross linking with modified polylysine as follows. Polylysine is first modified with a limited amount of succinic anhydride to convert a small fraction of the amino groups to carboxyl groups. The remaining majority of amino groups are protected by reaction with N-(tert-butoxycarbonyloxy)succinimide. The modified polylysine is linked to the substrate by carbodiimide chemistry employing carbodiimide and N-hydroxylsuccinimide. After linking the modified polylysine to the substrate, the protection groups are removed with acid to expose the amino groups. This step is repeated to amplify the number of amino functional groups to the desired density as described supra. At a desired level of density, these amino groups are converted to photoreactive groups by reacting with N-5-azido-2-nitrobenzoyloxysuccinimide (Pierce Chemical Company). Any residual amino groups are converted to carboxyl group by reacting with succinic anhydride to impart negative charges to reduce nonspecific interactions between microparticles and the substrate.

A population of microparticles is plated on the suitably derivatized surface. On the side of the wafer opposite the side in contact with the microparticles, a mask is applied, designed with holes for the desired location(s) for the population of microparticles. Photoreactive groups are activated by passing electromagnetic radiation of appropriate wavelength through the mask, thus initiating the coupling reaction to bind the microparticles to the plate. This process is repeated with different masks to associate other populations of microparticles with the desired locations on the substrate.

### Example 2

### Construction of a Microarray Using Microfluidic Methods

An exemplary apparatus of the invention (FIG. 1) comprises a plurality of microparticle reservoirs 10, 110, etc. Each reservoir contains a population of microparticles (derivatized with biotin on their surface) 11, 111, etc. each bearing a unique type of entity. Microparticles are released from a reservoir into channels 12, 112, etc. and moved along the channels to sites 13, 113, etc. Between each unique population of microparticles (i.e., between reservoirs 10 and 110, between channels 12 and 112, and between sites 13 and 113), is a barrier which allows the buffer (such as water) to pass but retains the microparticles within their respective reservoirs, channels and sites.

A population of microparticles is moved (individually or as a group), by microfluidics, from, for example, site 13 along a second channel 14 to site 15, and, from there, along channel 16 to site 17. At site 17, the population of microparticles, are aligned in close contact. The aligned microparticles are then moved along channel 18 to site 19, where the aligned microparticles are covalently or noncovalently cross-linked to one another to form a microparticle chain 20. Similarly, other microparticle chains 120, 220, etc. are made and moved to sites 119, 219, etc. At sites 19, 119, etc, in the buffer reservoir 2, buffer containing avidin is used to cross-link the microparticles.

The microparticle chains 20, 120, etc. are then transported from sites 19, 119, etc to sites 21, 121, etc. It is important to keep the chains extended and oriented in a fashion such that they can be stacked neatly. This is accomplished electromagnetically by designing microsized programmable electrodes or magnetic cores in this area to guide the movement of these strings of microparticles to line up and stack in an orderly fashion with other microparticle chains to form an array at sites 21, 121, etc.

Finally, the microparticle chains 20, 120, etc. are moved from sites 21, 121, etc. and deposited on the surface of a substrate 50, generating an array of addresses, wherein each address comprises a distinct entity.

### Example 3

### Use of a Microarray as a Template for the Production of Additional Microarrays

A microarray comprising an array of nucleic acid sequences is used as a template for the formation of additional microarrays, as follows.

The template microarray is constructed as follows: A plurality of unique nucleic acid sequences are bound to microparticles, and the microparticles are bound to a substrate such that each unique nucleic acid sequence occupies a unique address on the substrate. The 3′ end of the nucleic acid is coupled to the microparticle, and the 5′ end is distal to the microparticle. At the distal end of each nucleic acid sequence (i.e., the end farthest from the microparticle), a short common single stranded nucleic acid sequence is present, again with the 5′ end being distal to the microparticle.

The substrate for the new microarray is prepared as follows: A nucleic acid sequence complementary to the common sequence is bound to microparticles, with the 5′ end coupled to the microparticles, and the 3′ end distal. The microparticles are bound to a second substrate at distinct addresses.

The two arrays are placed in contact with each other, under ionic and buffer conditions which favor hybridization between the common sequences and their complements.

After hybridization between the common sequences and their complements, the arrays, still in proximity, are subjected to conditions favoring nucleotide polymerization by providing a DNA polymerase and deoxynucleoside triphosphate substrates under appropriate conditions of pH, ionic strength and cation concentration, as are known to those of skill in the art. The two arrays are then melted under appropriate conditions known to those of skill in the art to separate the two microarrays into microarrays each containing single stranded nucleic acids.

Polymerization will produce an ordered array of new nucleic acid sequences on the second microarray based on the nucleic acid sequences on the template microarray. This generates a microarray of nucleic acid sequences which are complementary to those on the template microarray, wherein the new nucleic acid sequences are attached to the complement of the common sequence at the 5′ end.

Although the foregoing invention has been described in some detail by way of illustration and example for purposes of clarity of understanding, it will be apparent to those skilled in the art that various changes and modifications can be practiced without departing from the spirit of the invention. Therefore the foregoing descriptions and examples should not be construed as limiting the scope of the invention.

What is claimed is:

1. A microarray comprising:
(a) a substrate, wherein the substrate is derivatized with either:
i) a first compound comprising a first functional group, and at least one layer of a cross-linking compound comprising multiple second functional groups, or
ii) a first compound comprising a first functional group, and a polymeric film comprising multiple second functional groups; and
(b) either:
i) a population of at least one entity of interest, wherein the population of at least one entity of interest is associated with a distinct address on the substrate through coupling of the entities and the second functional groups, or
ii) a population of microparticles, wherein the population of microparticles has at least one entity of interest coupled thereto, and wherein the population of microparticles is associated with a distinct address on the substrate through coupling of the second functional groups with the microparticles, such that the at least one entity of interest occupies a distinct address on the substrate.

2. The microarray of claim 1, wherein the microarray comprises more than one population of at least one entity of interest or population of microparticles.

3. The microarray of claim 1, wherein the at least one entity of interest is selected from the group consisting of nucleic acids, polypeptides, carbohydrates, cells, hormones, ligands, amino acids, lipids, fatty acids, small molecules, nucleosides, and nucleotides.

4. The microarray of claim 3, wherein the at least one entity of interest is a nucleic acid.

5. The microarray of claim 3, wherein the at least one entity of interest is a polypeptide.

6. The microarray of claim 3, wherein the at least one entity of interest is a cell.

7. The microarray of claim 1, wherein the at least one entity of interest is the product of a combinatorial chemistry procedure.

8. The microarray of claim 1, wherein more than one type of entity occupies a distinct address on the substrate.

9. The microarray of claim 8, wherein each address comprises at least one polypeptide and at least one nucleic acid.

10. A microarray comprising:
(a) a substrate; and
(b) a population of microparticles, wherein the population of microparticles is associated with a distinct address on the substrate, and wherein the population of microparticles has at least one entity of interest coupled thereto, the at least one entity of interest being selected from the group consisting of polypeptides, carbohydrates, cells, hormones, ligands, amino acids, lipids, fatty acids, and small molecules;
such that the at least one entity of interest occupies a distinct address on the substrate.

11. The microarray of claim 10, wherein the microarray comprises more than one population of microparticle.

12. The microarray of claim 10, wherein the at least one entity of interest is a polypeptide.

13. The microarray of claim 10, wherein the at least one entity of interest is a cell.

14. The microarray of claim 10, wherein the at least one entity of interest is the product of a combinatorial chemistry procedure.

15. A microarray comprising:
(a) a substrate; and
(b) a population of microparticles, wherein each microparticle is less than 1 μm in diameter, wherein the population of microparticles is associated with a distinct address on the substrate, and wherein the population of microparticles has at least one entity of interest coupled thereto,
such that the at least one entity of interest occupies a distinct address on the substrate.

16. The microarray of claim 15, wherein the microarray comprises more than one population of microparticles.

17. The microarray of claim 15, wherein the at least one entity of interest is selected from the group consisting of nucleic acids, polypeptides, carbohydrates, cells, hormones, ligands, amino acids, lipids, fatty acids, small molecules, antibiotics, nucleosides, and nucleotides.

18. The microarray of claim 17, wherein the at least one entity of interest is a nucleic acid.

19. The microarray of claim 17, wherein the at least one entity of interest is a polypeptide.

20. The microarray of claim 17, wherein the at least one entity of interest is a cell.

21. A microarray produced by a method comprising:
(a) providing a population of at least one entity of interest, wherein the entities are optionally coupled to microparticles;
(b) providing a substrate, wherein the substrate is derivatized with an activatible compound capable of coupling to the entities of interest or to the optional microparticles;
(c) contacting the population of entities with the substrate; and
(d) activating the activatible compound at the desired location(s) on the substrate,
such that the population of entities is coupled to the substrate in the desired location(s).

22. The microarray of claim 21, comprising more than one population of at least one entity of interest.

23. The microarray of claim 21, wherein the activation compound is activated by electromagnetic radiation.

24. The microarray of claim 21, wherein the desired location(s) are isolated by a mask comprising at least one orifice corresponding to the desired location(s) for the population of entities.

25. The microarray of claim 21, wherein the desired location(s) are isolated by a fiber optic beam.

26. The microarray of claim 21, wherein the desired location(s) are isolated by micromirrors.

27. The microarray of claim 21, wherein the entities are coupled to microparticles, and the substrate is derivatized with an activatible compound capable of coupling to the microparticles.

28. The microarray of claim 21, wherein the activatible compound is a photoreactive compound.

29. The microarray of claim 21, wherein the activatible compound is a heat activatible adhesive.

30. The microarray of claim 21, wherein the at least one entity of interest is selected from the group consisting of nucleic acids, polypeptides, carbohydrates, cells, hormones, ligands, amino acids, lipids, fatty acids, small molecules, nucleosides, and nucleotides.

31. A method for constructing a microarray, wherein the method comprises:
(a) providing a substrate, wherein the substrate is derivatized with either:
i) a first compound comprising a first functional group, and at least one layer of a cross-linking compound comprising multiple second functional groups, or
ii) a first compound comprising a first functional group, and a polymeric film comprising multiple second functional groups;
(b) providing either:
i) a population of at least one entity of interest, or
ii) a population of microparticles, wherein the population of microparticles has at least one entity of interest coupled thereto;
(c) localizing the population of entities or microparticles to a distinct address on a substrate; and
(d) associating the population of localized entities or microparticles to their distinct address on the substrate through coupling of the second functional groups to the entities of interest or to the microparticles.

32. The method of claim 31, wherein the at least one entity of interest is selected from the group consisting of nucleic acids, polypeptides, carbohydrates, cells, hormones, ligands, amino acids, lipids, fatty acids, small molecules, nucleosides, and nucleotides.

33. A method for constructing a microarray, wherein the method comprises:
(a) providing a population of at least one entity of interest, wherein the entities are optionally coupled to microparticles;
(b) providing a substrate, wherein the substrate is derivatized with an activatible compound capable of coupling to the entities of interest or to the optional microparticles;
(c) contacting the population of entities with the substrate; and
(d) activating the activatible compound at the desired location(s) on the substrate,
such that the population of entities is coupled to the substrate in the desired location(s).

34. The method of claim 33, comprising more than one population of at least one entity of interest.

35. The method of claim 33, wherein the activation compound is activated by electromagnetic radiation.

36. The method of claim 33, wherein the desired location(s) are isolated by a mask comprising at least one orifice corresponding to the desired location(s) for the population of entities.

37. The method of claim 33, wherein the desired location(s) are isolated by a fiber optic beam.

38. The method of claim 33, wherein the desired location(s) are isolated by micromirrors.

39. The method of claim 33, wherein the activatible compound is a photoreactive compound.

40. The method of claim 33, wherein the activatible compound is a heat activatible adhesive.

41. The method of claim 33, wherein the at least one entity of interest is selected from the group consisting of nucleic acids, polypeptides, carbohydrates, cells, hormones, ligands, amino acids, lipids, fatty acids, small molecules, nucleosides, and nucleotides.

42. A method of producing microarrays comprising nucleic acid sequences, comprising:
(a) providing a first microarray comprising:
(i) a first substrate;
(ii) a first population of at least one nucleic acid sequence, wherein the at least one nucleic acid sequence comprises a first nucleic acid hybridization sequence at the distal end of the nucleic acid sequence, wherein the first population of nucleic acid sequence(s) is optionally coupled to microparticle(s), and wherein the population of nucleic acid sequence(s) is associated with a distinct address on the first substrate;
(b) providing a second microarray comprising:
(i) a second substrate;
(ii) a population of second hybridization sequence(s), wherein the second hybridization sequence(s) is complementary to the first hybrization sequence(s), wherein the second population of hybridization sequence(s) are optionally coupled to microparticle(s), and wherein the population of hybridization sequence(s) is associated with a distinct address on the second substrate;
(c) contacting the first and second microarrays under hybridizing conditions, such that the first and second hybridization sequences hybridize;
(d) exposing the hybridized first and second microarrays to nucleotide polymerizing conditions, such that said at least one nucleic acid sequence from the first microarray is used as a template for the production of a complementary nucleic acid sequence on the second microarray.

43. The method of claim 42, wherein the first and second hybridization sequences are at least 5 nucleotides in length.

44. The method of claim 42, wherein the at least one nucleic acid sequence is a DNA sequence.

45. The method of claim 42, wherein the at least one nucleic acid sequence is an RNA sequence.

46. A method for producing multiple copies of a microarray on a single substrate, wherein the method comprises:
(a) providing a population of microparticles, wherein the population of microparticles has at least one entity of interest coupled thereto;
(b) providing a substrate for multiple copies of a microarray;
(c) localizing the population of microparticles to the substrate at the desired location(s) for each microarray to be produced on the substrate; and
(d) associating the population of microparticles to the substrate at the desired location(s) for each microarray to be produced on the substrate.

47. The method of claim 46, wherein the microparticles are associated with to the substrate by photoactivation.

48. The method of claim 46, wherein the microparticles are associated with to the substrate by a heat activatible adhesive

49. The method of claim 46, wherein the microparticles are localized to the desired location(s) by robotic dispensing.

50. The method of claim 46, wherein the microparticles are localized to the desired location(s) by microfluidics.

51. The method of claim 46, wherein the microparticles are localized to the desired location(s) with an electrical field.

52. The method of claim 46, wherein the microparticles are localized to the desired location(s) with a magnetic field.

53. The method of claim 46, wherein the at least one entity of interest is selected from the group consisting of nucleic acids, polypeptides, carbohydrates, cells, hormones, ligands, amino acids, lipids, fatty acids, small molecules, nucleosides, and nucleotides.
